(19) United States
(12) Patent Application Publication
(10) Pub. No.: US 2014/0147514 A1
(43) Pub. Date: May 29, 2014

(54) EXTRACTION OF VITAMINS AND MINERALS FROM PLANT MATTER

(71) Applicants: Ramaswamy Rajendran, Bangalore (IN); Kamala Rajendran, Bangalore (IN)

(72) Inventors: Ramaswamy Rajendran, Bangalore (IN); Kamala Rajendran, Bangalore (IN)

(21) Appl. No.: 14/131,326

(22) PCT Filed: Jul. 30, 2012
(86) PCT No.: PCT/IN2012/000530

§ 371 Date: Jan. 7, 2014

(30) Foreign Application Priority Data
Aug. 4, 2011 (IN) 2677/CHE/2011

(51) Int. Cl. A61K 31/525; A61K 33/26; A61K 36/752; A61K 36/31; A61K 36/75; A61K 36/61; A61K 31/355; A61K 31/375; A61K 31/51; A61K 31/4406; A61K 31/197; A61K 31/4415; A61K 33/06; A61K 33/00; A61K 33/04; A61K 36/53; A61K 36/85; A61K 36/899; A61K 36/185; A61K 36/24; A61K 33/30

(52) U.S. Cl. CPC A61K 31/525; A61K 33/30; A61K 33/26; A61K 36/752; A61K 36/31; A61K 36/75; A61K 36/61; A61K 31/355; A61K 31/375; A61K 31/51; A61K 31/4406; A61K 31/197; A61K 31/4415; A61K 33/06; A61K 33/00; A61K 33/04; A61K 36/53; A61K 36/85; A61K 36/899; A61K 36/185; A61K 36/24
USPC 424/641; 424/646; 424/725; 424/736; 424/755; 424/774; 424/777; 514/251; 514/458; 514/474

(57) ABSTRACT

A process for extracting vitamins and minerals from a first plant matter(s) is disclosed wherein the said first plant matter(s) is treated with acidic matter followed by extraction with water. Said treatment converts the water-insoluble vitamins and minerals into water-soluble forms leading to a more comprehensive extraction with better yields of the plant constituents. Suitable selection of the first and second plant matter(s) and their proportions yields an extract that is a ready-made formulation of the desired vitamins and minerals in RDA quantities. Other advantages are reduced processing times and processing steps. Extraction of guava fruits, guava leaves, holy basil leaves, lemon peels, amla fruits, annato seeds, Wrightia tinctoria, Lantana camara, bamboo shoots, mustard seeds, and curry leaves is described.

### CLAIM OF PRIORITY

Applicants are submitting these papers pursuant to §1893, of the Manual of PEP for entry of the national stage of the following PCT application, and this application makes reference to, incorporates the same herein, and claims all benefits accruing under 35 U.S.C. §365(c) of the PCT International application entitled EXTRACTION OF VITAMINS AND MINERALS FROM PLANT MATTER filed on 30 Jul. 2012 and duly assigned Serial No. PCT/IN2012/000530 and makes reference to, incorporates the same herein, and claims all benefits under 35 U.S.C. §119 from applications filed in the Indian Intellectual Property Office on 4 Aug. 2011 and there duly assigned Serial No. 2677/CHE/2011.

### BACKGROUND OF THE INVENTION

### Field of the Invention

This invention relates to the extraction of phytochemicals and nutrients from plant matter and more particularly to the extraction of phytochemicals and nutrients from a first plant matter(s) by employing water as a solvent, and comprising the application of one or more reacting compound(s) thereto such as to convert at least, one or more of the water-insoluble phytochemicals/nutrients therein into water soluble form(s), said reacting compound(s) being preferably applied in the form of a second plant matter(s).

Although plant nutrients are also phytochemicals, the distinction has been made here because this invention is particularly related to the extraction of nutrients such as vitamins and minerals contained in plant matter. The process of the invention is however, easily and simply applied to the extraction of constituents and phytochemical compounds of plants, other than the vitamins and minerals.

In the interests of conciseness, therefore, the further description hereinbelow is in the context of the extraction of said vitamins and minerals but this is without any limitation to the scope of the invention. Said plant vitamins and minerals together are referred to herein either by the term ‘plant nutrients’ or ‘nutrients’. The process of the invention is equally suitable for other nutrients such as, for example, proteins, fats, carbohydrates, to nutrients and antioxidants other than vitamins and minerals, and to odour, colouring, flavour, taste and other factors. Thus, the terms ‘nutrients’, ‘plant nutrients’, ‘plant constituents’ and ‘vitamins and minerals’ are all intended herein to have the maximum scope of including all constituents, principles and factors present in the plant matter.

About 20 vitamins, vitamin precursors and mineral nutrients are found in plant matter. Most plant matters contain a plurality of said nutrients.

Said nutrients are extracted from plant matter by means of solvents. When a solvent is applied to a plant matter in an extraction operation a solution is obtained containing one or more of the plant constituents. A set of desired nutrients usually do not have a common solvent and, as such, to obtain a set of nutrients from plant matter, two or more extractions become necessary. These extractions are referred to in the art as primary extractions and the extracts as primary extracts for the reason that each contains a plurality of the plant constituents in solution. Primary extracts and extractions are also referred to as crude extracts and crude extractions respectively and the solvents employed are referred to as primary or crude solvents.

A primary extraction may be done on fresh (unextracted) plant matter or a plant matter that has already undergone one or more extractions. A plant matter that has undergone one or more extractions is referred to herein as ‘spent plant matter’. The term is used herein for both partially and fully spent plant matter. The extracted plant matter arising at any stage in a multi-stage extraction process is referred to herein as ‘spent plant matter’ or ‘spent matter’. The meaning appropriate to the context may be taken.

The scope of the terms ‘extraction’, ‘extraction operation’ and ‘extraction step’ in the description and claims of this invention are intended to cover both single-stage extractions and multi-stage extractions.

Primary extracts are required to be subjected to fractional extraction procedures or other separation procedures to isolate the different nutrients. A fractional separation usually involves several secondary solvents for the purpose of isolating the different nutrients through fractionation/partitioning steps. An extraction of a set of nutrients and their isolation may thus involve conducting a plurality of primary extractions followed by fractional extraction procedures one for each primary extraction.

In prior art procedures, said nutrients are first isolated and thereafter the isolated nutrients are admixed to yield the formulations. Different formulations contain different combinations of said vitamins and minerals as required for different patient profiles, disease/disorders, health requirements and dietary standards and recommendations. Said isolated nutrients are admixed in the desired quantities and proportions to make different dietary, nutraceutical, medicinal and other formulations.

It will be observed from the above that the said isolation and remixing of nutrients in prior art processes for making formulations is wasteful of processing time, materials and energy. This drawback is removed in the process of the invention wherein a substantially ready-made formulation is obtained at the end of a single primary extraction operation using a single solvent.

In prior art, the isolated nutrients are optionally subjected to one or more processing operations such as, for example, crushing, grinding, milling, screening, sifting, mixing, homogenising and others and/or on the mixture. Such steps are eliminated in the process of the invention wherein said nutrient isolation operations are redundant.

There are a number of similar optional operations that are carried out on the formulated nutrient mixture in the prior art. Within the scope of the invention, the process of the invention may include any of such operations by way of finishing of the primary extract or for other purposes. Examples, of such optional additional operations are: crushing, grinding, pulverising, milling, screening, sifting, mixing, homogenising, agglomerating, drying, treating with excipients, treating with additives and others.

Sometimes, the set of primary extracts containing the desired nutrients in solution are subjected to solvent removal in prior art yielding a set of solid or semi-solid extracts which are then admixed to give the formulation. Although in this procedure said isolation of nutrients is not carried out, the drawback of requiring multiple extractions and multiple extracting solvents remains.

Conventional processes for said formulations, thus involve multiple extractions, multiple fractionation steps for nutrient isolation, multiple primary and secondary solvents and their handling, storage and recovery systems. Their main disadvantage is the systemic disadvantage inherent in a procedure that comprises firstly the isolation of a set of materials/compounds and subsequent remixing thereof.

This invention observes that if a way could be found such that all the desired nutrients could be extracted out from the plant matter by means of a single solvent in a single extraction step, there will be considerable cost benefits. Similarly, if the said process of isolation and re-mixing can be substituted by a process wherein the desired formulation is directly obtained at the end of the extraction step, there can be further advantages and cost benefits such as considerable energy saving and others. Such benefits will be in capital costs, running costs, materials, processing time and others. A significant benefit will be the elimination of a host of primary and secondary solvents, their use, storage and recovery. This is the basis of the process of the invention.

Even greater cost benefits can be realised if the said single common solvent can be water. This will avoid edibility and toxicity problems associated with the organic solvents normally employed in conventional extractions. Mostly, conventional extractions have toxicity issues to a greater or lesser extent. Extraction by water will yield an aqueous solution of the said nutrients, which will constitute a ready and administrable formulation in liquid form. It will be a ready-made mixture requiring substantially no processing before administrations to subjects. A solid form formulation can be easily obtained therefrom by the simple operation of water removal through any of the known procedures therefor such as evaporation, absorption, adsorption or others.

The solvents adopted in conventional procedures are usually one of the lower aliphatic alcohols, aliphatic and aromatic hydrocarbon solvents such as butanes, pentanes, hexanes, benzene and toluene, alcohols, ethers, ketones, esters, halogenated hydrocarbons such as ethylene dichloride, methylene dichloride, chloroform, carbon tetrachloride and others and mixtures thereof. Water is also used as a solvent with the limited object of extracting the water-soluble nutrients and not as a comprehensive solvent for all, or substantially all of the desired nutrients, as in the process of the invention.

By the adoption of water, as in the process of the invention, the drawback associated with the undesirable properties of the organic solvents used in prior art procedures is overcome. The undesirable properties associated with hydrocarbons, ethers, esters, alcohols and halogen-substituted solvents and other solvent or solvent mixtures are: inedibility, toxicity, carcinogenicity and mutagenicity. The residual levels of said prior art solvents in the extracted nutrients are therefore required to be very low particularly as these nutrients are required to be taken by subjects over long periods of time and many times on a lifelong basis. The costs of achieving such low residual solvent levels are high. This drawback is eliminated by the use of water as solvent in the process of the invention.

This invention provides a treatment process whereby one or more of the water-insoluble nutrients in the plant matter to be extracted are converted into forms that are water-soluble. It will be observed that this extends the extractability range of water allowing the extraction of many more of the nutrients in the plant matter. Indeed, by careful selection of process factors it is possible to extract out the full complement of nutrients in a plant matter(s) in a single extraction operation using single solvent, namely, water in the process of the invention. The process of the invention offers the scope for extracting any set of desired nutrients corresponding to a formulation through a single primary extraction operation to give a primary extract that substantially constitutes said formulation.

It will be observed that in this manner substantially the full set of nutrients present in a plant matter can be obtained in a ready-mixed, more or less formulated form upon the removal of the solvent from the extract solution. Such nutrient mixtures, whether in the form of solid, semi-solid or a concentrated solution, are directly administrable to subjects. They may be easily converted into various dosage forms. They are also very suitable intermediates for easy conversion into medicinal, food and nutraceutical and beverage products. They are also suitable for conversion into food and dietary preparations and supplements.

Thus, the basis of the process of the invention is the said treatment by means of which the role of water is transformed into a substantially universal solvent, able to extract substantially all, or the entire set of nutrients in a plant matter(s).

Said treatment of the invention has been devised such as to render one or more of said water-insoluble nutrients soluble and additionally to preferably render one or more of the water-soluble nutrients more water-soluble. Preferably, the treatment renders the said nutrients, water-soluble and/or water-insoluble into forms that have better biological and metabolic properties, such as, for example, increased activity and assimilability.

Examples of said nutrients that are water-insoluble and that can be rendered water-soluble by the treatment of the invention are the FSVs(fat-soluble vitamins), namely, vitamins A, D and K which are found in plants and plant-derived materials.

Adopting said treatment of the invention allows one to use water solvent to extract out all, or substantially all of the nutrients contained in any plant matter(s). Said treatment comprises reacting said water-insoluble and other nutrients with suitable reacting compounds to convert them into forms that are water-soluble. Similarly, water-soluble plant ingredients can be converted into forms that are more water-soluble. It can also be used to impart greater efficacy and activity to the nutrients.

It will be observed that considerable cost savings can be achieved in capital, labour and operating costs by the adoption of water as the common solvent for the set of all, or substantially all, said nutrients contained in a plant matter. By the choice of suitable said reacting compounds, one can favourably influence properties such as biological activity.

If the adoption of a particular said reagent(s) is advantageous from the extraction perspective but yields said converted forms that are not biologically active, or less active, then, within the scope of the invention one or more secondary conversion steps may be incorporated in the process of the invention with the object of imparting, or increasing, said activity.

A wide variety of methods are known in the art for contacting of the plant matter and solvent(s) and for said separation, all of which are within the scope of the invention. The particular nutrients extracted and the extent of extraction depends on such factors as the parameters of extraction, such as temperature, duration, degree of agitation, the size and other characteristics of the plant matter being extracted and others.

It is therefore the object of this invention to adopt a single solvent to extract substantially the entire set of nutrients in plant matters.

It is a further object of this invention to adopt water as the said single solvent and to provide said reacting compounds that convert one or more of said water-insoluble nutrients into water soluble forms.

A still further object of this invention is to devise said treatments such that one or more of the said water-soluble nutrients are converted into forms that are more water-soluble.

A still further object is to devise said treatment such that the said converted forms are more active biologically and metabolically.

A still further object of the invention is to obtain the set of desired said vitamins, nutrients and other constituents in a ready-mixed form and in the desired proportions such as for example, the RDA values following a single extraction step with a single extraction solvent, namely, water.

Other objects of the invention will become apparent from the description and claims of the specification.

The advantages of the process of the invention for extracting nutrients in plant matters are:
(i) the elimination of multiple solvents and multiple extraction procedures;
(ii) the adoption of water as the said single solvent for extracting substantially all the said nutrients;
(iii) the elimination of the fractional extraction procedures;
(iv) the elimination of solvent toxicity and other such adverse factors in the extract;
(v) getting substantially all said nutrients in a single extract solution, or as a single solid or semi-solid mixture by the evaporation of the solvent therefrom in reduced processing times, at lower costs and with lesser operating steps;
(vi) simplification of the extraction process by reduction of processing time, reduction of capital costs, operating costs and reduced requirement of materials;
(vii) the use of plant materials containing said reacting compounds instead of reagents for the said treatment of said first plant matter(s);
(viii) the simultaneous extraction of said nutrients contained in the said reacting-compounds containing second plant matter(s);
(ix) substantially total extraction of the vitamins and minerals from the said first and second plant matter(s), and
(x) operating flexibility.
   Other advantages will be evident from the further description hereinbelow.

According to the invention, therefore, there is provided a process for the extraction of vitamins and minerals and other constituents from a first plant matter(s) comprising the treating of said first plant matter(s) with reacting compound(s) for converting one or more of the water-insoluble vitamins and minerals and other constituents therein into water-soluble forms, thereafter extracting said first plant matter(s) with water, or a dilute extract coming from a previous or another extraction, to obtain an extract solution product comprising said vitamins and minerals and other constituents of said first plant matter(s) in solution.

Within the scope of the invention, said reacting compounds may be acidic, alkaline or others. Said compounds are preferably acidic and may be actual acids or other acidic compounds. They may be any of the mineral acids(inorganic acids) or organic acids within the scope of the invention. Preferably, they are plant-based organic acidic compound(s) which may be applied to the plant matter to be extracted in the form of plant matter. Said acidic compound(s) may be free acids and/or other acidic compound(s).

Said treatment with organic acidic compounds makes the said water-insoluble nutrients water-soluble. Suitable choice of the plant-based organic acid constituents can be made such that the water solubility of many of the water-soluble nutrients is increased and the rate of extraction enhanced making the extraction operation speedier.

Within the scope of the invention, said organic acids may be in the form of reagents. As would be observed from the description of the embodiments of the invention included herein, suitable selection of the acid containing plant matter can give better yields of said nutrient components and also provide other cost benefits. Suitable choice of said acid components can give said converted forms of the nutrients that are more metabolically active and absorbable.

More preferably, the free organic acids for said application are in the form of plant matter which is applied to said first plant matter(s) containing the vitamins and minerals and other constituents to be extracted.

Said plant matter comprising reacting compounds, for application to the said first plant matter(s) to produce said converted forms is referred to herein as the second plant matter(s).

The process of the invention involves applying said reacting compounds to the first plant matter(s) that contains the nutrients to be extracted. The application may be in the solid state wherein the two plant matters are brought into intimate contact. Suitable size reduction of the plant matter may be carried out for better contact. The objective is to increase the contact area. Other suitable preparatory operations may be carried out on the said first and second plant matters such as cutting, chopping, dicing, crushing, grinding, cleaning, washing, drying, blanching, macerating and others. The term ‘nutrients’ is intended to cover all said vitamins, minerals and other constituents of the first plant matter(s). Pulping of the two said plant matters and contacting the two pulp matters for the purpose of said treatment is also within the scope of the invention.

The two plant matters may be contacted in a liquid phase comprising a suitable liquid wherein the two plant matters are dispersed. Contacting may be enhanced by stirring/agitation or by fluidisation means. Within the scope of the invention, said contacting may be at ambient temperatures and pressures or under elevated temperatures and/or pressures. Hydrothermal cooking may also be adopted in the interests of better yields and for other cost benefits. Said liquid contacting phase may be any suitable liquid wherein the plant matter mixture is slurried. It may be the said adopted solvent or other liquid. Preferably, the contacting phase is water, the solvent adopted in the process of the invention.

If water is adopted as the contacting phase as well as the solvent, it will be observed that the said conversion and extraction by dissolution in the solvent will proceed side-by-side. Preferably, said first and second plant matters may be added to the water in that order but addition of the two matters the other way round is also within the scope of the invention. Forming of a semi-solid mass of the two plant matters with water, or other liquid medium, for carrying out said conversion is also within the scope of the invention. It may be noted that said contacting phase and the solvent for extraction may be the same or different within the scope of the invention

Within the scope of the invention, said treatment operation may precede the extraction operation or alternatively, the two operations may be carried out simultaneously as is the case in the arrangement wherein the two plant matter(s) first and second, are dispersed in the extraction solvent.

The extraction of the water-soluble components of the said first plant matter(s) or said second, or both may be carried out separately prior to carrying out said reaction(treatment) of the water-insoluble components within the scope of the invention. The process of the invention would then require another extraction operation so as to extract the converted said vitamins, minerals and other constituents.

Within the scope of the invention, each said first and second plant matter(s) may be a mixture of two or more species of plants. Said second plant matter that provides the acidic component(s) for said conversion may also be a source of said nutrients like the said first plant matter(s) which may also comprise said acidic(reacting) compounds within the scope of the invention.

That is, within the scope of the invention, the extract product of the invention may comprise nutrient components coming from both said first and second plant matter(s). Similarly, a part of the acidic compound(s) for said conversion can come from the said first plant matter(s) within the scope of the invention. It will be observed that the choice of the different species going into said first and second plant matter(s) and of the range of acidic materials available give one options to produce tailor-made mixtures of said nutrients having substantially pre-determined proportions of the nutrients and to organise said production in an optimised manner from the point of view of energy consumption, processing times and other production factors. The solution extract products obtained at the conclusion of said extraction operations in the process of the invention are directly administrable to subjects.

As would be noted, the process of the invention makes the conventional practice of the isolation of the nutrients followed by subsequent admixing of the separated nutrients for making of the formulations, redundant. In the process of the invitation, the panel of nutrients as present originally in the two plant matters is not disturbed and is brought out substantially intact, and as a ready mixture, into the extract of the invention. Said panel of nutrients can be obtained in the form of a concentrated solution form by removal of the solvent from the extract. Known means for solvent removal are available in the art, such as, for example, removal by evaporation, by absorption, adsorption and other processes. Further solvent removal can give the said panel in a solid or semi-solid form. Said solid or semi-solid mixtures and the said concentrated extracts are all referred to as extracts or extract products herein.

The terms ‘extract’ and ‘extract product’ of the invention are intended to include within the scope thereof the dilute extract solution that is obtained at the end of the extraction step in the process of the invention; any concentrated solution obtained upon removal of the solvent from a said dilute solution; the solid or semi-solid product obtained on extended removal of solvent from the dilute extract solution and a product obtained by further processing of said dilute, concentrated, solid or semi-solid product such as, for example, adsorption on excipients; crushing, grinding, milling, screening, sifting, blending, homogenising and others; agglomeration operations such as for example, tablet-making, pellet-forming and others; conversion into any of the known pharmaceutically acceptable salts; conversion to associate the product nutrients with any of the known pharmaceutically accepted carriers; conversion into other forms such as, for example, having greater efficacy and biological availability, conversion into any of the known dosage forms; topping-up of said nutrients/constituents; incorporation of additional said nutrients/constituents and/or of additives for colour, taste, flavour, bulk, texture, odour and others; and other such operations.

In the process of the invention, said process of admixing, that is addition of individual nutrients is limited to topping up of the nutrient levels in the extract of the invention to reach RDA(Recommended Daily Allowances) levels or other specified, or standard or recommendatory, levels and requirements. Additional nutrients, that is, nutrients not present in the original plant matter and therefore not present in the extract mass may also be added to build up the desired nutrient distribution in the formulations within the scope of the invention.

Within the scope of the invention, plant matter of two or more species may be combined to constitute the said first plant matter and similarly for the said second plant matter such that the resulting extract product, solid, semisolid or concentrated extract solution, comprises the required combination of said nutrients in the required amounts/proportions. Said required combination and amounts may be in accordance with any of the medicinal, nutritional and other standards and specifications. The said species going into said first and second plant matter(s) may be selected such as to give desired combinations of the said acidic reacting components and the components of the final extract and also give specific synergistic advantages such as more rapid extraction, said converted forms having greater efficacy and others.

Said first and second plant matter(s) may comprise the roots, leaves, fruits, flowers, stems, branches, bark, rhizomes or other plant parts or any mixture thereof within the scope of the invention. Said first and second plant matter(s) may be employed in the process of the invention as mixtures of the desired plant species or otherwise. Such an option provides another tool to favourably influence the process performance and economics and generate synergies.

As mentioned, the role of a said first or second plant matter(s) is not required to be exclusive within the scope of the invention. A said plant matter (first or second) may provide the said nutrient(s) or said organic acidic reacting compound(s) or both.

As mentioned, considerable cost saving can result if groups of said nutrients, and preferably all of said nutrients in a plant matter are extracted by a single solvent and the solvent removed to give a substantially finished mixture of the said nutrients. This eliminates a number of primary extraction and fractional extraction steps as also the said step of admixing the isolated nutrients.

The extract product of the invention contains substantially the full spectrum of nutrients present in the plant matter and is a ready intermediate for preparing the formulations and processing them into various dosage forms. The only processing required as far as their nutrient content is concerned is the said topping step and the addition of missing nutrients if any.

In cases where the spectrum of nutrients obtained from a plant matter(s) in the extract is adequate for a particular application, no topping and missing nutrient addition will be necessary. The invention provides the basis for extracting out all the principles in a plant matter(s). The extract in such a case can be considered to be the essence of the original plant matter extracted.

The process of the invention can faithfully bring out substantially the full spectrum of nutrients originally present in the plant matter extracted.

The term ‘extraction’ is used herein to refer to both the process of extraction as a whole and the extraction step(operation) therein. The meaning appropriate to the context may be taken. As mentioned, an extraction step may consist of a single extraction or plurality of stages of extraction within the scope of the invention.

Some of the mineral acids tried and found suitable by this invention for said conversion are: hydrochloric, sulphuric, phosphoric, nitric and perchloric acids. Some of the organic acids

similarly tried and found suitable for said conversion by this invention are succinic, citric, tartaric, acetic and ascorbic acids.

Said treatment with acidic components converts the said nutrients into salts, esters, alcohols and other compounds that are water-soluble.

This invention observes that their investigations show that adoption of organic acids in the form of plant matter containing the acids gives better extraction yields of the vitamins and minerals and also offers many other synergistic advantages and benefits. Within the scope of the invention, said organic acids may be added to the said first plant matter in reagent forms or in the form of said second plant matter. In general, the said acidic compound(s) for application to the said first plant matter may be either in the form of reagents and/or in the form of the appropriate plant matter(s) within the scope of the invention. As mentioned, the acidic component(s) may also come from the said first plant matter. Said reagent acidic compounds and plant-based acidic compounds may be free acids or otherwise.

The term ‘plant matter(s)’, said first, second or other, is intended to mean that the plant matter may also be a plurality of plant species. References to ‘plant matter’ herein are also intended to cover the option of a plurality of plant species.

It will be observed that the process of the invention will also extract nutrients from the said second plant matter(s) that contains the desired organic acid components required for said conversions. From the range of plant matter available for use as said acidic second plant matter(s), the process of the invention offers a number of selections. Selection of the said first and second plant matter(s) may be made such as to achieve specific technical and techno-economic objectives. The process of the invention offers numerous options from the point of view of selecting the combination of the species forming the said first and second plant matter(s) to maximise yields; to obtaining desired combinations of said nutrients in desired proportions; reduce processing times; to minimise post-extraction processing; and other cost and material benefits.

The advantages of providing acidity in the form of said second plant matter(s), and optionally additionally in the form of said first plant matter(s) is that plant matter is safe and non-toxic as established by the long usage of centuries thereof by traditional societies as food and/or medicines.

In a particular situation, the resulting converted nutrients may be in water-soluble forms but one or more thereof may not be biologically(metabolically) active. In such a situation, the process of the invention can incorporate a further step after the water extraction step wherein either the extract solution or the solid or semi-solid extract is treated with a suitable reagent(s) to convert the said biologically inactive components into active forms. By a suitable choice of said acid components one can obtain more active forms and forms that can be more easily assimilated.

More preferably, this invention provides for admixing a first vitamin and mineral containing plant matter to be extracted with a second plant matter containing the desired organic acids either in the free form or in the form of other acidic compounds, and then contacting the said mixture of first and second plant matters with water to extract the vitamins and minerals of the said first plant matter and of the second plant matter.

Within the scope of the invention, said first plant matter may comprise a mixture of two or more different plant matters comprising the vitamins and minerals to be extracted. Within the scope of the invention, said organic acid contacting and the extracting operation by water may be combined.

In one embodiment, therefore, this invention provides a method for the extraction of one or more vitamin or mineral components in a first plant matter comprising;
(i) providing said first plant matter(s);
(ii) providing a second plant matter(s) comprising one or more organic acidic compounds that are free organic acids or otherwise;
(iii) admixing the said first and second plant matter(s); and
(iv) extracting the said mixture of first and second plant matter(s)s with water to yield an extract solution containing said vitamins and minerals and other constituents coming from the said first and second plant matter(s).

Within the scope of the invention, said first and second plant matter(s) may be subjected to one or more preliminary(preparatory) operations such as of cutting, dicing, macerating, crushing, grinding, screening, washing, drying, blanching and others. Within the scope of the invention, steps (iii) and (iv) may be a combined operation. The process steps described above are amenable to different arrangements and order. Similarly, several variants can be visualised by persons in the art. Such other variants, order and arrangements of the abovementioned steps are within the scope of the invention.

The investigations of this invention reveal that some of the acid containing plant matter that was found suitable for adoption as a said second plant matter(s) are:
(i) Amla fruits(Emblica officinalis) which contain citric and ascorbic acids,
(ii) Tamarind fruits which contain tartaric and cinnamic acids,
(iii) Guava fruits which contain ascorbic acid,
(iv) Lemon peels containing folic acid,
(v) Hibiscus subdarifa containing hydroxyl citric acid,
(vi) Hibiscus rosasinensis containing oxalic acid, citric acid, tartaric acid,
(vii) Garcinia cambogia fruits containing hydroxyl citric acid,
(viii) Garcinia indica fruits containing hydroxyl citric acid,
(ix) Rosemary leaves containing rosmarinic acid,
(x) Acerola fruits containing ascorbic acid,
(xi) Orange and lemon peels containing citric acid,
(xii) Acai berries containing oleic acid, linoleic acid, palmitic acid and ascorbic acid,
(xiii) Ocimum sanctum leaves containing ursolic acid, stearic acid, palmitic acid, ascorbic acid and chlorogenic acid, and
(xiv) Lagerstroemia speciosa leaves containing corosolic acid.

As mentioned, said acidification of the first plant matter can be optimised with regard to yield and other considerations by a suitable choice of one or more of the abovementioned acidic-compound-containing second plant matters.

Based on the investigations carried out by the present inventors, a few examples of said first plant matter that can be extracted by the process of the invention to yield an extract containing a mixture of substantially all the vitamins and minerals therein through a single extraction with water are:
(i) Bamboo containing silica, calcium and phosphorous,
(ii) Curry leaf containing iron, magnesium and other vitamins and minerals,
(iii) Mustard seeds containing selenium and other vitamins and minerals,
(iv) Lantana camara herb which contains magnesium, calcium, copper and other vitamins and minerals,
(v) Wrightia tinctoria containing calcium, magnesium, potassium, copper and other vitamins and minerals,
(vi) Equisetum arvensis containing silica, calcium and copper,
(vii) Urtica dioica containing silica,
(viii) Garcinia cambogia peels containing calcium, sodium and other vitamins and minerals,
(ix) Guava leaf containing zinc and other vitamins and minerals,
(x) Ocimum sanctum leaves containing zinc, manganese, sodium calcium, and
(xi) Moringa leaf containing chromium and calcium.

It may be mentioned that the scope of the invention is not limited by the examples provided herein.

The process(method) of the invention yields an extract that is a mixture of the vitamins and minerals contained in the two plant matter(s), said first and second. The proportions in this formulation of can be simply and easily adjusted to the desired values by the process of topping-up and by the admixture thereof with other vitamins and minerals so as to meet RDA or other requirements. A ready-made, directly administrable formulation that is easily converted into any dosage form and that is a very convenient intermediate for further conversion is the major advantage of this invention. This advantage can be further increased and enlarged by suitable choice of said plant matters and their nutrient and acidic constituents, from the wide range of available options some of which are listed hereinabove.

Within the scope of the invention, the roles played by the said first and second plant matters need not be exclusive. A said first plant matter whose nutrients are being extracted may contain acidic components that can play the said role of conversion of the said water-insoluble nutrients contained in the first and/or second plant matters.

This invention observes that the adoption of said acidic compounds in the form said second plant matter or otherwise increases the nutrient yield in the extract.

As mentioned, this invention does not lay down exclusive roles for said first and second plant matters. If a said plant matter is labelled a first plant matter it is implied that its function as a nutrient source is primary but its role as a source for said acidic constituents for reacting with the said nutrient compounds may be as important as the said nutrient source role. If a said plant matter is labelled a second plant matter it is implied that its function as a provider of said acidic

reactants is primary while its role as a nutrient source may be as important as said providing of acids/acidic compounds.

In the process of the invention, the desired profile as regards the said nutrients components in the extract of the invention may be obtained by a suitable choice of species forming the two plant matters, said first and second, of the invention. Desired proportions of the said nutrient components in the extract of the invention can be obtained by adjusting the proportions of the said first and second plant matters to be extracted including the individual species therein. This results in an extract(solid, semi-solid or dilute or concentrated solution) that is a very suitable and convenient intermediate for conversion into dosage forms in so far as the step of admixing of the said nutrient components is substantially eliminated in the process of the invention.

Required proportions of the said nutrients in the extract of the invention can be obtained by selecting suitable plant species to constitute said first and second plant matters and taking them in suitable proportions.

The art provides different methods for contacting of the two plant matters. Similarly, various methods are available in the art for contacting the said first and second plant matters with the solvent water. Numerous process variants are possible considering the said contacting methods, extracting methods, procedures and layouts, the order of the various processing steps in particular, the said treatment(contacting) and extraction operations. Having a plurality of plant species in the said first and second plant matters introduces further process variants based on different combinations of mixing of the species, extracting of the species, the order of applying the different acidic reactants and others. All such variants are within the scope of the invention.

As mentioned, different extraction operation flow patterns are possible with regard to the spent matter and extract solution streams. In such flow patterns found in multi-stage extraction operations, dilute extract streams are often used to contact plant matter in the role of solvents. Thus, the term ‘water solvent’ in the description and claims herein also covers the use of such dilute extract solutions as extracting solvents. The process of the invention is easily adaptable for single stage and multi-stage extraction and for batch, semi-continuous and continuous operation.

In order to provide a clearer understanding of the invention and without limitation to the scope of the invention, a few embodiments thereof are described hereinbelow.

### Embodiment 1

Guava fruits were taken. They contain the sensitive vitamins B1, B2, B5 and B6. The fruits were taken up for extraction without drying as drying partly decomposes the said vitamins.

Holy basil leaves were dried and taken for extraction.

Lemon peels containing folic acid were dried and taken for extraction.

The said first plant matter comprised a mixture of the three abovementioned components: guava fruits, holy basil leaves and lemon peels.

The holy basil leaves and lemon peels constituted said second plant matter. The second plant matter in this embodiment is a mixture of species. The acidic components therein were the oleanolic, ursolic, rosemarinic acids of holy basil leaves and the citric and ascorbic acids contained in the lemon peels.

About 99 kg. of fresh guava fruits together with about 0.5 kg. of holy basil leaves and about 0.5 kg. of dried lemon peels after extraction with water yielded a mixture B1, B2, B3, B5, B6 and B9 vitamins.

The extract product was analysed and the composition determined. It was also subjected to various tests to ascertain various parameters and properties. Said composition, parameters and properties are presented in Table I which also gives the required composition, characteristics and properties. It will be observed that there is good agreement between the required values/properties and that obtained in the extract of the invention.

The vitamin levels were in general accordance with the RDA levels.

Table I gives a comparison of the extract of the abovementioned embodiment of the invention with the required values and parameters. Column 2 of the table gives the desired specifications and Column 3 gives the values obtained in the said extract of the embodiment. The quantities of the B-vitamins therein are substantially in accordance with RDA requirements. The table indicates excellent conformation of the said extract of the embodiment with the desired parameters.

TABLE I
Comparison of the extract of the embodiment of the invention with the
required specifications.

| | | | Testing |
|---|---|---|---|
| Physical Analysis | Specification | Actual Values | method |
| Appearance and Color | Greyish Brown to | Complies | Visual |
| | Brown powder | | |
| Identification | Positive | Complies | HPTLC |
| Taste | Bitter & irony | Complies | Organoleptic |
| Odor | Characteristic | Complies | Organoleptic |
| Solubility | Soluble in water | Complies | USP XXIII |
| Loss on Drying | NMT 6% w/w | 3.3% | USP XXIII (IR) |
| Particle sizel | 98% min. thro’ 20 | 100% | USP XXIII |
| | mesh | | |
| Assay of Actives | Specification | Result | Test Method |
| Vitamin Bl (Thiamin) | NLT 22 mg/g (2.2%) | 24 mg/g | HPLC |
| Vitamin B2 (Riboflavin) | NLT 20 mg/g (2%) | 22 mg/g | HPLC |
| Vitamin B3 (Niacin) | NLT 34 mg/g (3.4%) | 36 mg/g | HPLC |
| Vitamin B5 (Pantothenic | NLT 38 mg/g (3.8%) | 40 mg/g | HPLC |
| acid) | | | |
| Vitamin B6 (Pyridoxine | NLT 22 mg/g (2.2%) | 24 mg/g | HPLC |
| Vitamin B9 (Folic acid) | NLT 700 mcg/g | 755 mcg/g | HPLC |
| | (0.07%) | | |
| Microbiology | Specification | Result | Test Method |
| Total Plate Count | 1000 cfu/gram max | Complies | USP XXIII |
| Yeast and Mold | 100 cfu/g max | Complies | USP XXIII |
| Coliforms | Absent | Absent | USP XXIII |
| Salmonella | Absent | Absent | USP XXIII |
| E. coli | Absent | Absent | USP XXIII |
| Pseudomonas aeruginosa | Absent | Absent | USP XXIII |
| Staphylococcus aureus | Absent | Absent | USP XXIII |
| Chemical Impurities | Specification | Result | Test Method |
| Heavy Metals | NMT 10 ppm | Complies | USP XXIII |
| Arsenic as As | NMT 0.5 ppm | Less than 0.001 PPM | USP XXIII |
| Mercury as Hg | NMT 1 ppm | Less than 0.01 PPM | USP XXIII |
| Cadmium as Cd | NMT 1 ppm | Less than 0.01 PPM | USP XXIII |
| Lead as Pb | NMT 1 ppm | Less than 0.01 PPM | USP XXIII |
| Pesticides residue | Absent | Absent | USP XXIII |

| | |
|---|---|
| Remarks | The extract complied with the required specifications |

### Embodiment 2

In this embodiment, a mixture of guava fruits and amla fruits was extracted with water. The two fruits constituted said first and second plant matter respectively. Higher yields of the vitamins were obtained. By taking suitable proportions of the two plant matters a very useful formulation of vitamins B and C was obtained that matched the RDA requirements. The role of guava fruits was substantially exclusively that of a said first plant matter and that of amla was a combination of the role of the said first and second plant matters.

### Embodiment 3

In this embodiment, minerals were extracted. Guava leaf contains zinc. Direct extraction of the leaf with water or other solvents was found to be poor.

Under the process of the invention, the guava leaf was extracted together with about 1-2% by wt. of amla fruit pulp. The guava leaf and the amla fruit pulp were the said first and second plant matter and the solvent was water.

The extraction of zinc was much better. The yield was about 10% with a zinc content of 4% by wt.

The yield in the comparative example based on the prior art procedure mentioned was found to be poor, the yield being only about 0.6% and a zinc content of only 0.1% only.

### Embodiment 4

In this embodiment, said first plant matter comprises a mixture of Wrightia tinctoria, Lantana camara, bamboo shoots, mustard seeds, and curry leaves. About 1-2% by wt of amla fruit pulp was added to the abovementioned mixture. Said amla fruit pulp constituted the said second plant matter.

In the absence of amla fruit pulp the extraction of the minerals was very poor. The organic acids in the amla fruit pulp gave a considerably increased yield of the minerals.

The minerals extracted were calcium from Wrightia tinctoria, magnesium from Lantana camara, silica from the bamboo shoots, selenium from the mustard seeds and iron from the curry leaves.

Quantities of the various components of the said first plant matter mixture were chosen such that about 400-600 mg of the dried extract contained the RDA amounts of the abovementioned minerals.

### Embodiment 5

This embodiment is a process of extracting fresh annato seeds(said first plant matter together with fresh amla fruits(said second plant matter) to give an extract containing vitamin E.
(i) Fresh annato seeds were collected, crushed in a stainless steel multimill to get seed pulp.
(ii) Fresh amla fruits were collected, crushed in a stainless steel multimill to get
(iii) amla pulp.
(iv) About 495 kgs of annato seed pulp with 5 kg of amla fruit pulp were charged into an extractor.
(v) The extractor comprised a stainless steel vessel of about 5000 L capacity provided with an agitator system and a surrounding jacket for heating by steam.
(vi) About 2000 L of water were charged into the extractor.
(vii) The extractor contents were maintained at about 50-65 deg C. by heating with steam.
(viii) The extraction was carried out for a period of about 6 hours.
(ix) During the extraction, the extract was recirculated across the bed containing the annato and amla pulp mixture in the extractor.
(x) At the end of the extraction period, the extract was collected in a tank. The extract was denoted A. About 1500 L of extract was obtained.
(xi) The spent pulp was subjected to another extraction by the procedure as outlined herein. The extract was withdrawn at the end of the extraction period and collected in a tank. This extract was denoted B. About 1500 L extract was obtained.
(xii) The spent pulp after the two extractions mentioned herein was further extracted in a third extraction with about 1500 L water. The temperature was maintained at about 50-65 deg C. during extraction.
(xiii) About 1500 L extract was obtained. This extract is denoted C.
(xiv) Extracts A and B were both separately concentrated in concentrators at about 50-65 deg C. down to a volume of about 150 L each. Falling film evaporators were used. The two extracts contained the Vitamin E tocopherols in aqueous solution.
(xv) Extract C was used as solvent and charged into extractor for extracting said pulp mixture as mentioned herein.
(xvi) The two concentrated extracts A and B were combined giving about 300 L of concentrated extract. This was filtered in a stainless steel Nutsche type filter using ‘Hyflosupercel’ as filter aid.
(xvii) The clear filtrate obtained from step (xvi) was spray dried in a stainless steel spray drier at about 170 to 180 deg C. to yield the vitamin E tocopherols in a powder form.
(xviii) The vitamin E powder was ground in a stainless steel multimill and then sifted in a stainless steel sifter to a particle size of about 40-80 mesh. The sifted material was blended in an octagonal blender for about one hour to get a homogeneous powder material. The quantity of the product(yield) was about 50 kgs.

The analysis of the vitamin E powder product is given in Table II below. Table II gives a comparison of the required properties/characteristics with the properties/characteristics obtained in the extract made by the process of the invention. The required parameters/properties/characteristics are given in Column 2 and those of the extract made by the process of the invention in Column 3.

The process of the invention, including each of the embodiments described hereinabove, can be simply and easily adapted for batch, continuous or semi-continuous operation.

TABLE II
Comparison of the extract of the embodiment of the invention with the
required specifications.

| | | | Testing |
|---|---|---|---|
| Physical Analysis | Specification | Actual Values | method |
| Appearance and Color | Orange brown powder | Complies | Visual |
| Identification | Positive | Complies | HPTLC |
| Odor | Characteristic | Complies | Organoleptic |
| Taste | Characteristic | Complies | Organoleptic |
| Particle size | NLT 98% thro’ 20 mesh | Complies | USP XXIII |
| Loss on Drying | NMT 6% w/w | 3.1% | USP XXIII |
| | | | (IR) |
| Assay of Actives | Specification | Result | Test Method |
| Assay of Vitamin E | NLT 15% | 15.8% | HPLC |
| Chemical Impurities | Specification | Result | Test Method |
| Total Heavy Metals | NMT 10 ppm | Complies | USP XXIII |
| Lead | NMT 5 ppm | Less than 0.01 PPM | USP XXIII |
| Cadmium | NMT 1 ppm | Less than 0.01 PPM | USP XXIII |
| Arsenic | NMT 3 ppm | Less than 0.01 PPM | USP XXIII |
| Mercury | NMT 1 ppm | Less than 0.001 PPM | USP XXIII |
| Residual pesticides | Absent | Absent | USP XXIII |
| Microbiology | Specification | Result | Test Method |
| Total Plate Count | 1000 cfu/gram max | Complies | USP XXIII |
| Yeast and Mold | 100 cfu/g max | Complies | USP XXIII |
| E.coli | Absent | Absent | USP XXIII |
| Coliforms | Absent | Absent | USP XXIII |
| Salmonella | Absent | Absent | USP XXIII |
| Pseudomonas | Absent | Absent | USP XXIII |
| aeruginosa | | | |
| Staphylococcus aureus | Absent | Absent | USP XXIII |

| | |
|---|---|
| Remarks | The extract complied with the required specifications |

Embodiments and variations other than described herein above are feasible by persons skilled in the art and the same are within the scope and spirit of this invention.

What is claimed is:

1. A process for the extraction of vitamins and minerals and other constituents from a first plant matter(s) comprising the treating of said first plant matter(s) with reacting compound(s) for converting one or more of the water-insoluble vitamins and minerals and other constituents therein into water-soluble forms, thereafter extracting said first plant matter(s) with water, or a dilute extract coming from a previous or another extraction, to obtain an extract solution product comprising said vitamins and minerals and other constituents of said first plant matter(s) in solution.

2. The process for extraction of vitamins and minerals and other constituents from a first plant matter(s) as claimed in the preceding claim 1 wherein said reacting compound(s) comprise acidic compound(s) that is either a free acid(s) and/or other acidic compound(s).

3. The process for extraction of vitamins and minerals and other constituents from a first plant matter(s) as claimed in the preceding claim 2 wherein the said acidic compound(s) is in the form of a second plant matter(s) comprising one or more acidic-compound containing plant species.

4. The process for extraction of vitamins and minerals and other constituents from a first plant matter(s) as claimed in any of the preceding claims 1 to 3 wherein the said treatment and extraction operations are carried out substantially simultaneously in the solvent water by dispersal/slurrying of said first and second plant matter(s) in said solvent, said plant matter(s) being preferably in pulped or powdered form.

5. The process for extraction of vitamins and minerals and other constituents from a first plant matter(s) as claimed in any of the preceding claims 1 to 4 wherein said second plant matter(s) comprises Lemon peels and optionally and additionally other acidic-component containing plant matter(s).

6. The process for extraction of vitamins and minerals and other constituents from a first plant matter(s) as claimed in the preceding claim 5 wherein said first plant matter(s) comprises Guava fruits, Holy basil leaves and Lemon peels, and the said second plant matter(s) comprises Holy basil leaves and Lemon peels.

7. The process for extraction of vitamins and minerals and other constituents from a first plant matter(s) as claimed in the preceding claim 6 wherein the said extract solution product comprises at least vitamins B1, B2, B3, B5, B6 and B9 and optionally other said vitamins and minerals and other constituents.

8. The process for extraction of vitamins and minerals and other constituents from a first plant matter(s) as claimed in the preceding claim 7 wherein the said vitamin, mineral and other constituents in the said extract solution product substantially correspond to the RDA(Recommended Daily allowance) values or according to any of the other medical, nutritional and therapeutic standards/requirements.

9. The process for extraction of vitamins and minerals and other constituents from a first plant matter(s) as claimed in any of the preceding claims 1 to 4 wherein the said second plant matter(s) comprises Amla fruit matter and optionally other acidic-component containing plant matter(s).

10. The process for extraction of vitamins and minerals and other constituents from a first plant matter(s) as claimed in the preceding claim 9 wherein said first plant matter(s) comprises Guava fruits and Amla fruits and the said second plant matter(s) comprises Amla fruit matter.

11. The process for extraction of vitamins and minerals and other constituents from a first plant matter(s) as claimed in the preceding claim 10 wherein the said extract solution product comprises at least vitamins B and C and optionally other said vitamins and minerals and other constituents.

12. The process for extraction of vitamins and minerals and other constituents from a first plant matter(s) as claimed in the preceding claim 11 wherein the said vitamin, mineral and other constituents in the said extract solution product substantially correspond to the RDA(Recommended Daily allowance) values or according to any of the other medical, nutritional and therapeutic standards/requirements.

13. The process for extraction of vitamins and minerals and other constituents from a first plant matter(s) as claimed in the preceding claim 9 wherein said first plant matter(s) comprises Guava leaves and the said second plant matter(s) comprises Amla fruit matter.

14. The process for extraction of vitamins and minerals and other constituents from a first plant matter(s) as claimed in the preceding claim 13 wherein the said extract solution product comprises at least zinc and optionally other said vitamins and minerals and other constituents.

15. The process for extraction of vitamins and minerals and other constituents from a first plant matter(s) as claimed in the preceding claim 14 wherein the said vitamin, mineral and other constituents in the said extract solution product substantially correspond to the RDA(Recommended Daily allowance) values or are according to any other medical, nutritional and therapeutic standards/requirements.

16. The process for extraction of vitamins and minerals and other constituents from a first plant matter(s) as claimed in the preceding claim 9 wherein said first plant matter(s) comprises Wrightia tinctoria, Lantana camara, bamboo shoots, Mustard seeds and Curry leaves and the said second plant matter(s) comprises Amla fruit matter.

17. The process for extraction of vitamins and minerals and other constituents from a first plant matter(s) as claimed in the preceding claim 16 wherein the said extract solution product comprises at least Calcium, Magnesium, Silica, Selenium and Iron and optionally other said vitamins and minerals and other constituents.

18. The process for extraction of vitamins and minerals and other constituents from a first plant matter(s) as claimed in the preceding claim 17 wherein the said vitamin, mineral and other constituents in the said extract solution product substantially correspond to the RDA(Recommended Daily allowance) values or are according to any of the other medical, nutritional and therapeutic standards/requirements.

19. The process for extraction of vitamins and minerals and other constituents from a first plant matter(s) as claimed in the preceding claim 9 wherein said first plant matter(s) comprises Annato seeds and the said second plant matter(s) comprises Amla fruit matter.

20. The process for extraction of vitamins and minerals and other constituents from a first plant matter(s) as claimed in the preceding claim 19 wherein the said extract solution product comprises at least vitamin E and optionally other said vitamins and minerals and other constituents.

21. The process for extraction of vitamins and minerals and other constituents from a first plant matter(s) as claimed in the preceding claim 20 wherein the said vitamin, mineral and other constituents in the said extract solution product substantially correspond to the RDA(Recommended Daily allowance) values or are according to other medical, nutritional and therapeutic standards/requirements.

22. The process for extraction of vitamins and minerals and other constituents from a first plant matter(s) as claimed in any of the preceding claims 1 to 21 wherein said treatment further comprises rendering one or more water-soluble vitamins, minerals and other plant constituents in the said first and second plant matter(s) into forms that are more water-soluble and/or that are biologically and metabolically efficacious and active or that possess enhanced levels of said efficacy and activity.

23. The process for extraction of vitamins and minerals and other constituents from a first plant matter(s) as claimed in any of the preceding claims 1 to 22 wherein said extract solution product is optionally processed further according to one or more of the undermentioned steps:
(i) removal of the solvent from the extract solution by any of the known procedures such as, for example, evaporation to yield a concentrated extract product;
(ii) removal of the solvent from the extract solution by any of the known procedures such
as, for example, evaporation to yield a solid, or semi-solid extract product;
(iii) drying of the product from (i) or (ii) by any of the known drying procedures to yield a
solid form extract product and optionally subjecting the said product to any of the known finishing operations such as crushing, grinding, powdering, milling, sieving, sifting, mixing, blending, homogenising and others;
(iv) producing a solid form extract by adsorbing the extract on a suitable excipient and adopting a suitable binder as required;
(v) converting the extract into any of the known dosage forms;
(vi) converting the vitamins, minerals and other nutrients in the extract into any of the known pharmaceutically acceptable salts;
(vii) adopting any of the known pharmaceutical carriers for the said vitamins, minerals and other nutrients of the extract;
(viii) incorporation of additives such as for colour, texture, taste, flavour, bulk, odour and others in the said extract;
(ix) topping-up of one or more of said vitamins, minerals and other nutrient levels in the said extract and/or addition of one or more of other said vitamins, minerals and other nutrients such as to bring the extract into correspondence with any desired standard or specification, or for other purposes;
(x) incorporating additives and conversion into a medicinal, food or nutraceutical product;
(xi) any of the known processes/procedures for enhancing the efficacy and/or assimilability of one or more said vitamins, minerals and other nutrients in the extract; and
(xii) any other known processing step.

24. The process for extraction of vitamins and minerals and other constituents from a first
plant matter(s) as claimed in any of the preceding claims 1 to 23 wherein the said first and second plant matter(s) are optionally subjected to one or more of preparatory operations such as, for example, washing, cleaning, dicing, cutting, washing, macerating, blanching, drying, crushing, grinding, milling, screening, and others.

25. A process for extraction of vitamins and minerals and other constituents from a first plant matter(s) substantially as hereindescribed.

26. The extract product produced by a process for extraction of vitamins and minerals and other constituents from a said first plant matter(s) and/or optionally said second plant matter(s) as claimed in any of the preceding claims 1 to 25.
